(19) United States
(12) Patent Application Publication
(10) Pub. No.: US 2026/0026697 A1
(43) Pub. Date: Jan. 29, 2026

(54) METHODS AND SYSTEMS FOR DISPLAYING SLEEP AND GRIND DATA

(71) Applicant: Asesso Health Inc., Pleasanton, CA (US)

(72) Inventors: William C. Cliff, Pleasanton, CA (US); Abhijit Limaye, San Jose, CA (US)

(21) Appl. No.: 19/282,478

(22) Filed: Jul. 28, 2025

Related U.S. Application Data
(60) Provisional application No. 63/676,272, filed on Jul. 26, 2024.

(51) Int. Cl. A61B 5/0205; A61B 5/00; A61B 5/024; A61B 5/11; G16H 50/30

(52) U.S. Cl. CPC A61B 5/0205; A61B 5/02438; A61B 5/11; A61B 5/4557; A61B 5/4809; A61B 5/4812; A61B 5/6814; A61B 5/6824; A61B 5/6826; A61B 5/742; G16H 50/30; A61B 2560/0462; A61B 2562/0219

(57) ABSTRACT

Methods and systems for displaying sleep and grind data are disclosed. In an example, a method involves displaying, on a display of a computing device, time-aligned sleep data and grind data, wherein the sleep data was generated from heart rate data collected from a wearable heart rate sensor, and the grind data was generated from motion data collected from a wearable motion sensor.

### CROSS-REFERENCE TO RELATED APPLICATION

This application claims priority to provisional U.S. Patent Application Ser. No. 63/676,272, filed Jul. 26, 2024, which is incorporated by reference herein.

### BACKGROUND

Temporomandibular Joint Dysfunction (TMD) and bruxism, often interconnected conditions, affect a significant portion of the population. TMD refers to dysfunction in the temporomandibular joint (TMJ) connecting the jaw to the skull, while bruxism involves the grinding or clenching of teeth, usually during sleep. TMD (sometimes referred to as TMJ) and/or bruxism can lead to various symptoms, including chronic headaches, muscle pain, damage to the teeth, and disrupted sleep patterns. The constant grinding of teeth associated with bruxism can exacerbate TMD symptoms, creating a cycle of discomfort and poor sleep. It is estimated that many millions of people suffer from TMD, with a notable percentage also experiencing bruxism, illustrating the widespread impact of these disorders on daily life and overall well-being.

### SUMMARY

Methods and systems for displaying sleep and grind data are disclosed. In an example, a method involves displaying, on a display of a computing device, time-aligned sleep data and grind data, wherein the sleep data was generated from heart rate data collected from a wearable heart rate sensor, and the grind data was generated from motion data collected from a wearable motion sensor.

In an example, the sleep data is graphically represented in multiple different sleep stages on the display and the grind data is displayed on a sleep stage-specific basis.

In an example, the sleep data is graphically represented in multiple different sleep stages on the display and the grind data is displayed on a per-sleep stage basis.

In an example, the sleep data is graphically represented as sleep stages that include light, deep, REM, and awake and the grind data is displayed on a sleep stage-specific basis.

In an example, the sleep data is graphically represented as sleep stages that include light, deep, REM, and awake and the grind data is displayed as a numerical representation of grinds per sleep stage.

In an example, the time-aligned sleep data and grind data includes graphical indicators that link the sleep data to the grind data in time.

In an example, the sleep data includes time-series sleep stage data, the grind data includes time-series grind data, the time-aligned sleep data and grind data is generated by matching times of the time-series sleep stage data with times of the time-series grind data.

In an example, the time-series sleep stage data is displayed as sleep stages of light, deep, REM, and awake, and the time-series grind data is displayed with a sleep stage that has a matching time.

In an example, grind data from the time-series grind data is assigned to a sleep stage that has a matching time.

In an example, the grind data corresponds to a number of grinds that are detected from the motion data.

In an example, the grind data is displayed as a numerical representation of grinds per sleep stage.

In an example, the grind data is displayed as a grind score per sleep stage.

In an example, the grind data is displayed as a total number of grinds for a night of sleep.

In an example, the grind data is displayed as a grind score for a night of sleep.

In an example, the time-aligned sleep data and grind data were generated from heart rate data and motion data that were collected in time intervals that overlap with each other.

In an example, the wearable heart rate sensor is a finger worn device and the wearable motion sensor is a chin worn device.

In an example, the wearable heart rate sensor is a wrist worn device and the wearable motion sensor is a chin worn device.

In another example, a non-transitory computer readable medium comprising instructions to be executed in a computer system is disclosed. The instructions, when executed in the computer system, perform a method that involves displaying, on a display of a computing device, time-aligned sleep data and grind data, wherein the sleep data was generated from heart rate data collected from a wearable heart rate sensor, and the grind data was generated from motion data collected from a wearable motion sensor.

A system is also disclosed. The system includes a display, a processor, and memory with instructions stored thereon, wherein the instructions when executed by the processor cause time-aligned sleep data and grind data to be displayed on the display, wherein the sleep data was generated from heart rate data collected from a wearable heart rate sensor, and the grind data was generated from motion data collected from a wearable motion sensor.

In another example, a method involves displaying, on a display of a computer device, a comparison between 1) sleep data and grind data corresponding to a period when a dental splint was not being used by a person, and 2) sleep data and grind data corresponding to a period when a dental splint was being used by the person, wherein the sleep data was generated from heart rate data collected from a wearable heart rate sensor and is associated with a splint presence indicator (SPI) that corresponds to when the heart rate data was collected from the wearable heart rate sensor, the grind data was generated from motion data collected from a wearable motion sensor and is associated with an SPI that corresponds to when the motion data was collected from the wearable motion sensor, and the comparison is generated using the associated SPIs.

In an example, the sleep data is graphically represented as multiple different sleep stages on the display and the grind data is displayed on a sleep stage-specific basis.

In an example, the sleep data is graphically represented as multiple different sleep stages on the display and the grind data is displayed on a per-sleep stage basis.

In an example, the sleep data is graphically represented as multiple different sleep stages on the display and the grind data is displayed as a number of grinds per sleep stage.

In an example, the sleep data is graphically represented as multiple different sleep stages on the display and the grind data is displayed as a number of grinds per sleep stage, wherein the sleep stages includes light, deep, REM, and awake.

In an example, the sleep data is graphically represented as multiple different sleep stages on the display and the grind data is displayed on a sleep stage-specific basis, and the comparison between 1) sleep data and grind data corresponding to a period when a dental splint was not being used by a person, and 2) sleep data and grind data corresponding to a period when a dental splint was being used by the person that is displayed on the display of the computer device includes a graphical indication of a change in an amount of time spent in each sleep stage and a graphical indication of a change in a number of grinds counted in each sleep stage.

In an example, the comparison between 1) sleep data and grind data corresponding to a period when a dental splint was not being used by a person, and 2) sleep data and grind data corresponding to a period when a dental splint was being used by the person includes data points on a graph that have a marking that is indicative of whether the dental splint was not being used by the person or was being used by the person.

In an example, the SPIs are generated from a manual input into the computer device.

In an example, the SPIs are generated from presence data from a splint charging case.

In an example, the wearable heart rate sensor is a finger worn device and the wearable motion sensor is a chin worn device.

In an example, the wearable heart rate sensor is a wrist worn device and the wearable motion sensor is a chin worn device.

In an example, a non-transitory computer readable medium comprising instructions to be executed in a computer system is disclosed. The instructions, when executed in the computer system, perform a method involving displaying, on a display of a computer device, a comparison between 1) sleep data and grind data corresponding to a period when a dental splint was not being used by a person, and 2) sleep data and grind data corresponding to a period when a dental splint was being used by the person, wherein the sleep data was generated from heart rate data collected from a wearable heart rate sensor and is associated with a splint presence indicator (SPI) that corresponds to when the heart rate data was collected from the wearable heart rate sensor, the grind data was generated from motion data collected from a wearable motion sensor and is associated with an SPI that corresponds to when the motion data was collected from the wearable motion sensor, and the comparison is generated using the associated SPIs.

An example of a method is also disclosed. The method involves associating sleep data, which was generated from heart rate data collected from a wearable heart rate sensor, with a splint presence indicator (SPI) that corresponds to when the heart rate data was collected from the wearable heart rate sensor, associating grind data, which was generated from motion data collected from a wearable motion sensor, with an SPI that corresponds to when the motion data was collected from the wearable motion sensor, and displaying, on a display of a computing device, a comparison between sleep data and grind data collected when the associated SPI corresponds to a splint not being used and sleep data and grind data collected when the associated SPI corresponds to a splint being used.

Other aspects in accordance with the invention will become apparent from the following detailed description, taken in conjunction with the accompanying drawings, illustrated by way of example of the principles of the invention.

### BRIEF DESCRIPTION OF THE DRAWINGS

FIG. 1 depicts an example of an environment in which time-aligned sleep data and grind data can be generated and then displayed on the display of a computer device such as a smartphone.

FIG. 2 illustrates an example of a process for generating time-aligned sleep data and grind data that can be displayed on the display of a computer device such as a smartphone.

FIG. 3A is an example graph of time-series sleep data that was generated from heart rate data that was collected from a person over a night of sleep.

FIG. 3B is an example graph of time-series grind data that was generated from motion data that was collected from a person over a night of sleep.

FIG. 3C illustrates an example of time-aligning the time-series sleep data and the time-series grind data from FIGS. 3A and 3B.

FIG. 3D depicts an example of a set of time-aligned sleep data and grind data.

FIG. 4 is an example of a graphical representation of the time-aligned sleep data and grind data in the form of a bar graph.

FIG. 5 is another example of a graphical representation of the time-aligned sleep data and grind data in the form of a bar graph.

FIG. 6 is another example of a graphical representation of the time-aligned sleep data and grind data in the form of a bar graph.

FIG. 7 is another graphical representation of the time-aligned sleep data and grind data in the form of a sleep score and a grind score.

FIG. 8 illustrates an example of a process for generating comparison data that can be displayed on the display of a computer device such as a smartphone.

FIG. 9 depicts an example of multiple sets of time-aligned sleep data, grind data, and a splint presence indicator that correspond to multiple different nights of sleep for a person.

FIG. 10 illustrates an example of a process for generating a graphical representation of comparison data.

FIG. 11 is an example of a graphical representation of comparison data.

FIG. 12 is another example of a graphical representation of comparison data.

FIG. 13 is another example of a graphical representation of comparison data.

FIG. 14 depicts a computer device such as a smartphone that includes a processor, memory, a communications interface, and a user interface.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

As stated above, TMD and bruxism, often interconnected conditions, affect a significant portion of the population. TMD refers to dysfunction in the joint connecting the jaw to the skull, while bruxism involves the grinding or clenching of teeth, usually during sleep. TMD and/or bruxism can lead to various symptoms, including chronic headaches, muscle pain, damage to the teeth, and disrupted sleep patterns. The constant grinding of teeth associated with bruxism can exacerbate TMD symptoms, creating a cycle of discomfort and poor sleep. Treatments for bruxism and/or TMD may include stress management, dental interventions like dental splints or mouth guards, and lifestyle changes to reduce symptoms and prevent further damage to the teeth.

Advances in sensor technology have led to a class of consumer wearable sensors that are being widely used to track sleep. A wearable sleep tracking device is commonly worn on a finger as a ring or on a wrist as a watch or strap and uses a heart rate sensor to collect heart rate data that is translated into sleep data. For example, most consumer sleep trackers are typically designed to identify and track sleep in terms of the time spent in four different sleep stages, commonly characterized as light, deep, rapid eye movement (REM), and awake. To help the user of such a sleep tracker understand the sleep data that is generated, an application, or “App,” that is executed on a smartphone is typically provided with the sleep tracking device to collect and process the sleep data. The sleep tracker App is also configured to present the sleep data in a way that is indicative of the amount of time spent in each sleep stage. For example, the sleep data may be displayed on a smartphone as a bar graph that graphically represents the amount of time spent in each different sleep stage throughout a night of sleep. The sleep data can be presented to a user on a per-day basis or aggregated over, for example, a week, a month, or a year and the graphical display of the sleep stage data can be very helpful to understand the sleep data. Although sleep trackers are able to provide sleep data to users that is displayed on a per-sleep stage basis, relationships between bruxism and sleep, in particular, between bruxism and the amount of time spent in each sleep stage are not well known or understood, especially by the people that suffer the consequences of bruxism and/or TMD.

Advances in sensor technology have also enabled minimally invasive wearable motion sensors that are specifically designed to track motion of the jaw (e.g., mandibular movements) that occur during sleep. The motion data collected from such wearable motion sensors has been used to, for example, identify sleep disturbances that correspond to conditions such as sleep apnea.

Although wearable sleep tracking devices provide users with useful information about sleep patterns and new wearable motion sensors can identify sleep disturbances that correspond to sleep apnea, relationships between bruxism and sleep patterns are not easy to identify and understand. In view of the above, it has been realized that sleep data generated from heart rate data collected from a wearable heart rate sensor and bruxism data (e.g., grinding data) generated from motion data collected from a wearable motion sensor can be displayed on a display of a computer device in a time-aligned manner to provide a person with an easy way to understand the relationship between bruxism and sleep, which can be useful in their own personal health journey. In an example, the sleep data is graphically represented in multiple different sleep stages on the display of the computer device and the grind data is displayed on a per-sleep stage basis. In one particular example, the sleep data is graphically represented as sleep stages that include light, deep, REM, and awake and the grind data is displayed as a number of grinds that occurred during each sleep stage.

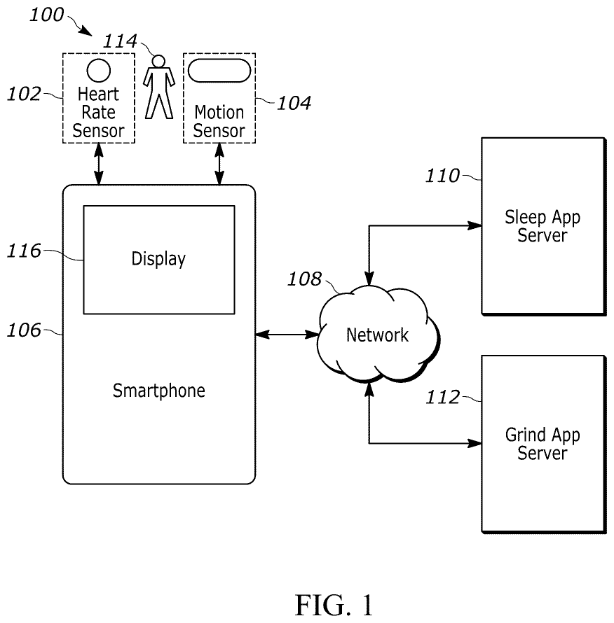

FIG. 1 depicts an example of an environment 100 in which time-aligned sleep data and grind data can be generated and then displayed on the display of a computer device such as a smartphone. In the example of FIG. 1, the environment includes a heart rate sensor 102, a motion sensor 104, a smartphone 106, a network 108, a sleep App server 110, and a grind App server 112. The heart rate sensor 102 may be in the form of a wearable sleep

tracking device such as, for example, a wearable finger ring, a wearable smartwatch, or a wearable wrist strap that is worn by a person 114. The wearable sleep tracking device may employ, for example, an optical sensor, or sensors, to monitor heart rate, heart rate variability, oxygen saturation (SpO2), and/or respiration rate as is known in the field. The wearable sleep tracking device may also include sensors for monitoring motion and/or body temperature to collect data that may be used to generate sleep data. Some examples of wearable sleep tracking devices are offered by OURA®, GARMIN®, FITBIT®, and WHOOP® to name a few. Although some examples of a wearable sleep tracking device are provided, other wearable health tracking devices may be used to collect data that can be used to generate sleep data.

The motion sensor 104 may be in the form of a wearable device that is worn at or near the chin of the person 114 to detect motion of the jaw and/or head of the person. In an example, the motion sensor includes an accelerometer that detects acceleration in the x, y, and z directions. The motion sensor may use other types of sensors to detect motion, such as a gyroscope. Although such a motion sensor can be worn at any point in a day or during any activity, it is anticipated that the motion sensor will be worn at night during a period in which the person expects to be sleeping. For example, the motion sensor may be attached at or near the chin of the person using an adhesive tape just before the person goes to bed and then the motion sensor can be removed soon after the person wakes up and gets out of bed. In an example, while not in use, the motion sensor may be docked in a case that is specifically designed to hold the motion sensor along with a dental splint and to download motion data from the motion sensor while the motion sensor is docked in the case. In an example, an App that executes on the smartphone 106 is associated with the motion sensor and the case and is configured to download the motion data from the case to the App running on the smartphone.

In an example, the motion sensor 104 is attached to the chin of the person 114. However, in another example, the motion sensor can be attached at another location on the person, such as at the side of the jaw. Additionally, another type of sensor may be used to generate the motion data. For example, a sensor may be able to detect changes in an electric parameter that corresponds to motion of the jaw and/or to movement of a muscle that controls jaw movement. Additionally, the motion sensor may include more than one sensor in which sensor data from each sensor is used to generate the motion data.

The smartphone 106 is a handheld communications device that includes at least one processor, memory, a communications interface, and a user interface such as a display 116. In an example, the smartphone has stored thereon a sleep App and a grind App. In one example, the sleep App is provided by the maker of the sleep tracking device (e.g., heart rate sensor 102) and the grind App is provided by the maker of the motion sensor 104, although the sleep App and the grind App could be provided independent of any device.

Although the computer device is described as a smartphone 106 in one example, the computer device may be a different type of computer device that is able to display the time-aligned sleep data and grind data. For example, the computer device may be a pad computer, a desktop computer, a laptop computer, or some other computing device.

In an example, the sleep App that executes on the smartphone 106 works in conjunction with the wearable sleep tracking device to collect heart rate data that is generated from the heart rate sensor 102 of the wearable sleep tracking device and to generate sleep data from the collected heart rate data. In an example, the sleep App executed on the smartphone works in conjunction with the sleep App server 110 to generate and maintain sleep data. The sleep App may be part of a health App stored on the smartphone that processes other data such as a number of steps in a day, stress, activity tracking, etc.

In an example, the grind App that executes on the smartphone 106 works in conjunction with the motion sensor 104 to collect motion data that is generated from the motion sensor and to generate grind data from the collected motion data. In an example, the grind App executed on the smartphone works in conjunction with the grind App server 112 to generate and maintain the grind data. Generating grind data from the motion data may involve identifying individual instances of grinds and keeping a database of the grinds that includes a timestamp associated with each grind. In general, an instance of a grind involves a clenching of the jaw (e.g., as a result of contraction of the masseter muscle) that causes at least some of the upper teeth and lower teeth to forcefully contact each other. In an example, motion data that is identified as being indicative of a grind, or indicative of grinding, has certain characteristics or a signature or a profile. For example, motion data that is indicative of grinding may include detected accelerations that exceed a particular threshold. In another example, motion data that is indicative of grinding may include detected accelerations that exceed a particular threshold while also being below a different threshold that is indicative of head movements. There are many different ways that grinds, grinding, or grind events can be gleaned from the motion data. Although examples of motion characteristics that are indicative of a grind, or indicative of grinding, are described, other motion characteristics, signatures, or profiles, of motion data that are indicative of a grind, or indicative or grinding, are possible.

In one example, the grind App provides grind data as a count of the number of individual grinds. In another example, the grind App provides grind data in terms of grind events. For example, a grind event may be a period of time in which grinding was detected, such as intermittent grinding sessions that may last anywhere from a few seconds to a few minutes. In another example, a grind event may be a single grind or certain number of grinds. In other examples, the grind App may provide grind data that characterizes grinding in terms of an amount of time spent grinding or a severity of grinding, e.g., light, moderate, severe, and/or a color coded characterization, green for light to no grinding, yellow for moderate grinding, and red for severe grinding. In another example, the grind data may be provided as a grind score, or as a time-series of grind scores. For example, a numerical score may be calculated for grind events that is a function of a number of grinds detected over a certain time interval. In an example, time-series grind data is a time-series of grind scores in which each score represents a magnitude of grinding during a particular time interval. As indicated above, the grind data generated from the motion data may characterize grinding in various different ways. In an example, it is desirable that the characterization of grinding can be understood by a person, especially so that the person can understand the magnitude and/or intensity of the grinding.

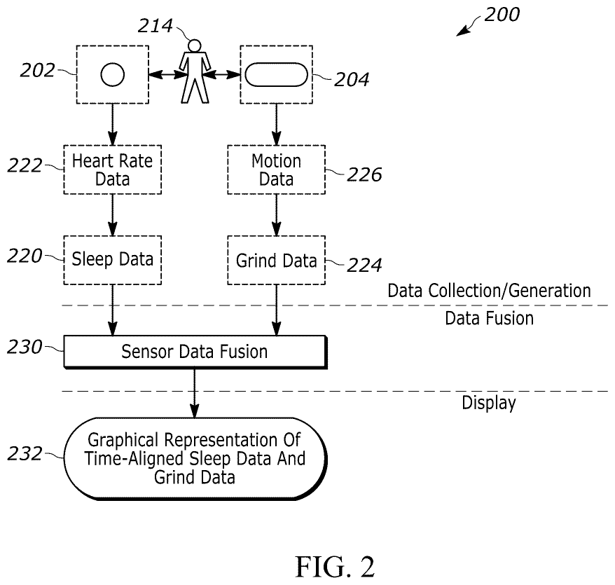

FIG. 2 illustrates an example of a process 200 for generating time-aligned sleep data and grind data that can be displayed on the display of a computer device such as a smartphone. With regard to sleep data 220, heart rate data 222 is generated from the heart rate sensor 202 of a wearable sleep tracking device that is worn by the person 214 as described above. The heart rate data that is collected by the heart rate sensor can be used to generate the sleep data, for example, as described above by a sleep App that is associated with the heart rate sensor. In an example, the sleep data includes time-series data that identifies a time-series of sleep stages. For example, the time-series sleep data may include time intervals marked by start times and end times with a sleep stage (e.g., light, deep, REM, awake) that corresponds to each time interval. In another example, the time-series sleep data may include fixed time intervals marked by a start time and an end time and a sleep stage that corresponds to each fixed time interval. As illustrated in FIG. 2, the sleep data is made available for a sensor data fusion process.

There are various different ways that sleep data 220 can be generated from the collected heart rate data 222. In an example, each different brand of wearable sleep tracking device may have its own unique way of generating sleep tracking data. For example, wearable sleep tracking devices offered by OURA®, GARMIN®, FITBIT®, and WHOOP® may each have a different way of generating sleep data from heart rate data. Further, the wearable sleep tracking devices, and their corresponding sleep App, may identify different stages of sleep and/or may present the sleep data in different ways.

With regard to grind data 224, motion data 226 is generated from the motion sensor 204 that is worn by the person 214. The motion data that is collected by the motion sensor can be used to generate grind data, for example, as described above by a grind App that is associated with the motion sensor. In an example, the grind data includes time-series grind data that identifies a time-series of grinds with each identified grind having a corresponding timestamp (e.g., YYYY-MM-DD hh: mm: ss), which identifies the time at which the grind occurred. In another example, the time-series grind data includes a time-series of fixed time intervals with each fixed time interval having a start time and an end time and a corresponding number of grinds that were identified within the fixed time interval (e.g., start time, end time, number of grinds). As illustrated in FIG. 2, the grind data is also made available for the sensor data fusion process.

In an example, a sensor data fusion process 230 involves time-aligning the sleep data 220 and the grind data 224 and generating a data set that can be converted to a graphical representation 232 of the time-aligned sleep data and grind data for display on a display of the computer device (e.g., the display on the smartphone). In one example, the sensor data fusion process is implemented at the grind App server (FIG. 1, 112) and in another example, the sensor data fusion process is implemented at the smartphone (FIG. 1, 106), e.g., by the grind App. In another example, the sensor data fusion process may be distributed over multiple computer devices.

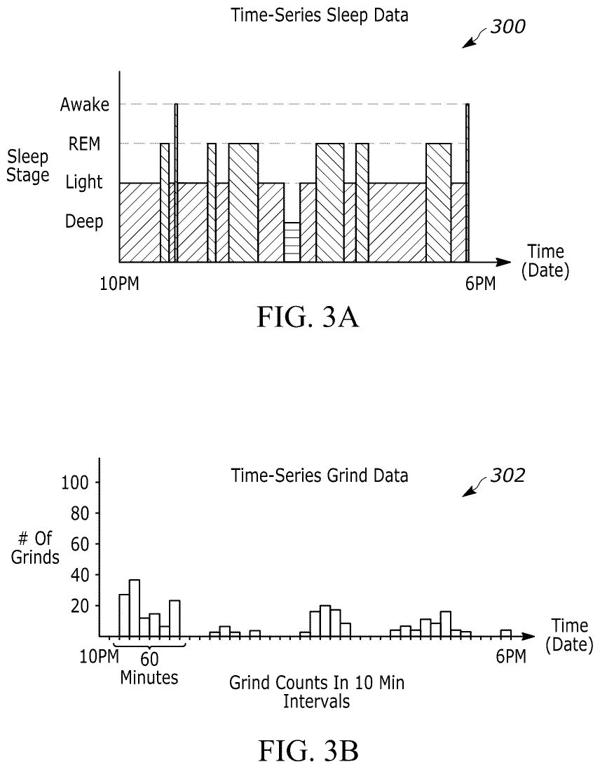

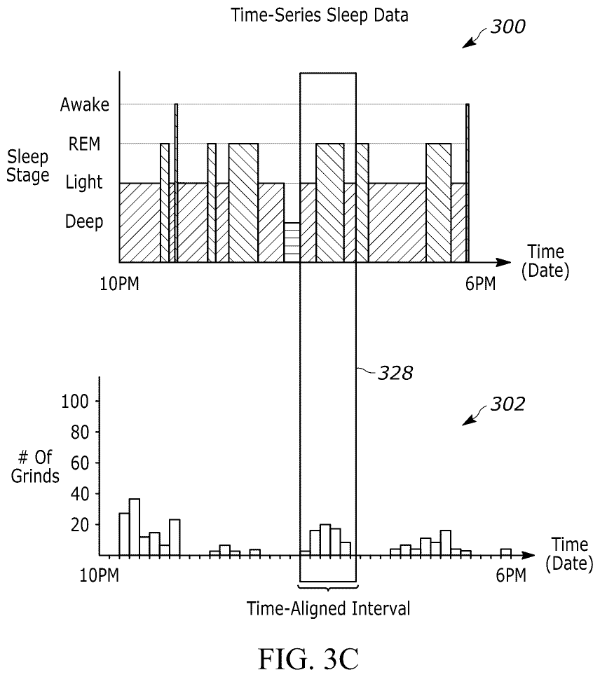

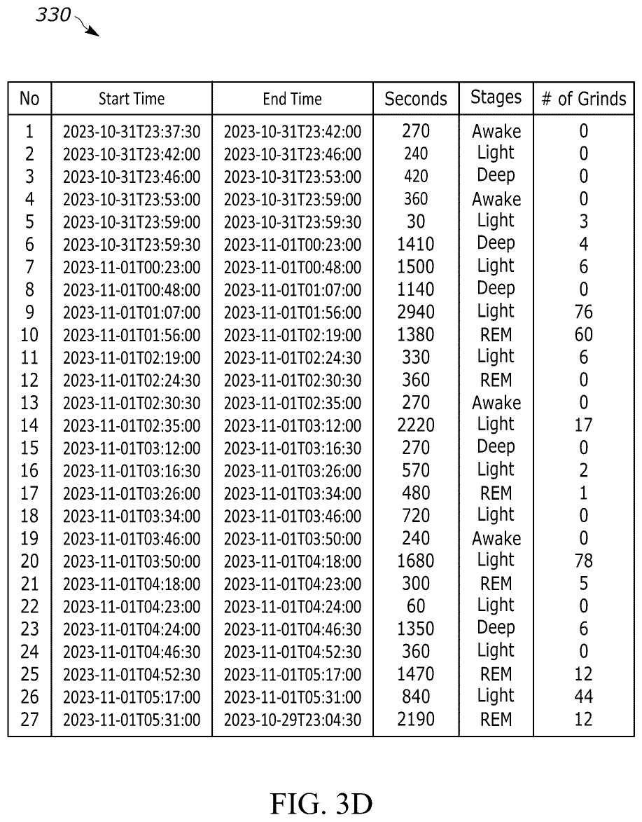

An example of the sensor data fusion process is described below with reference to FIGS. 3A-3D.

FIG. 3A is an example graph 300 of time-series sleep data that was generated from heart rate data that was collected from a person over a night of sleep. In particular, FIG. 3A is a bar graph of a time-series of sleep stages from one night of sleep starting at 10 PM at night and ending at 6 AM the next morning. As indicated on the graph, time is on the x-axis and sleep stage is on the y-axis. In an example, the sleep data is generated by a sleep App that corresponds to a wearable sleep tracking device that includes a heart rate sensor as described above. The time-series sleep data may include a start time, an end time, and a corresponding sleep stage for each time interval of a particular sleep stage. In another example, the sleep stage data is obtained from the sleep App server using an API although the sleep data could alternatively be accessed directly from the smartphone. Although the time-series sleep data is depicted in FIG. 3A as a bar graph for explanation purposes, the time-series sleep data could be generated and stored in a format such as a comma-separated values (csv) file.

FIG. 3B is an example graph 302 of time-series grind data that was generated from motion data that was collected over a night of sleep. In particular, FIG. 3B is a bar graph of a time-series of grind counts from one night of sleep starting at 10 PM at night and ending at 6 AM the next morning. As indicated on the graph, time is on the x-axis and number of grinds is on the y-axis. In the example of FIG. 3B, grind counts are aggregated over fixed time intervals, such as over 10 minute intervals although other fixed time intervals, either shorter or longer than 10 minutes, are possible. In an example, the time-series grind data includes a start time, an end time, and a grind count for each time interval. Although the time-series grind data is depicted in FIG. 3B as a bar graph for explanation purposes, the time-series grind data could be generated and stored in a format such as a “csv” file. Additionally, although the grind data is described in terms of grind counts (e.g., a number of grinds), the grind data may be characterized by grind scores, or grind events, as described above.

Once time-series sleep data and time-series grind data such as that described with reference to FIGS. 3A and 3B, respectively, has been obtained (e.g., by the grind App server), a process of sensor data fusion can be implemented by, for example, the grind App server, although the sensor data fusion could be implemented by a different computer, or by a combination of computers. In an example, the sensor data fusion process involves time-aligning the time-series sleep data and the time-series grind data. FIG. 3C illustrates an example of time-aligning the time-series sleep data and the time-series grind data from FIGS. 3A and 3B. In particular, the box 328 represents the time alignment of an interval of the sleep data with an overlapping interval of the grind data. In an example, time-aligning the time-series sleep data and the time-series grind data involves matching a sleep stage to a number of grinds over an overlapping time interval (e.g., over the same time interval). As illustrated in FIG. 3C, the interval of time aligned sleep data and grind data includes three different sleep stages that lasted for slightly over 60 minutes with a particular start time and end time and counts of grinds over the same time interval, which has the same start time and the same end time. In an example in which the time-series sleep data is organized by sleep stage and the time-series grind data is organized by fixed time intervals, the process of time-aligning the sleep data and the grind data may involve matching the fixed time intervals of the grind data to a corresponding (in time) sleep stage. For example, the process involves stepping through each sleep stage in chronological order and finding the count of grinds that corresponds to the time interval of the particular sleep stage. In some cases, an interval boundary of the grind data may not align exactly with a boundary of a sleep stage interval and the grind counts can be apportioned to specific sleep stages based on timestamps of the grinds. In an example, the “time” of an event (e.g., the start time of a sleep stage, the end time of a sleep stage, or the “time” corresponding to a grind) is recorded as a timestamp, which includes a date and time (e.g., on a 24 hour clock) of the day. A common notation of a timestamp is: YYYY-MM-DD hh: mm: ss. An example of a particular timestamp is: 2023 Oct. 31 T23:37:30, which corresponds to Oct. 31, 2023 at 11:37:30 PM.

Although in the example of FIG. 3C, the sleep data and grind data are aligned over the same time interval, in other examples, the time-aligned sleep data and grind data may have a substantial overlap in time, but the sleep data and the grind data may also have slightly different start times and/or end times. For example, it may be that start times and end times of the time intervals of sleep data and grind data may be off by a few seconds or a few minutes.

FIG. 3D depicts an example of a set of time-aligned sleep data and grind data 330. As shown in FIG. 3D, the set of time-aligned sleep data and grind data includes a column for entry numbers, a column for start times, a column for end times, a column for total number of seconds in the time interval, a column for the sleep stage that corresponds to the time interval, and a column for the number of grinds that corresponds to the time interval. In the example, the time intervals correspond to continuous intervals of time in which the person stayed in one sleep stage, with the sleep stages being light, deep, REM, and awake. Thus, in the example of FIG. 3D, the time intervals are not fixed time intervals. Rather, the length of each time interval depends on the sleep pattern of the person wearing the sleep tracking device. The set of time-aligned sleep data and grind data may be generated using, for example, the process described above with reference to FIGS. 3A-3C. In the example, the time-aligned sleep data and grind data corresponds to a night of sleep for a person, from when the person falls asleep to when the person wakes up to start a new day. The time-aligned sleep data and grind data may be stored as, for example, a csv file although other formats are possible. Although one example of a set of time-aligned sleep data and grind data is described with reference to FIG. 3D, the set of time-aligned sleep data and grind data may be expressed, stored, and/or managed in other forms or formats. Additionally, although the grind data is described in terms of a number of grinds (e.g., a grind count), the grind data may be generated and stored in other ways, e.g., by grind scores, or grind events, as described above.

In one example, the time-aligned sleep data and grind data may be displayed to a user on a display of a computer device in a format that appears similar to that shown in FIG. 3D. However, the format shown in FIG. 3D may not be easy to digest by the user and/or may not be easy to draw insights from. Therefore, with reference back to FIG. 2, the time-aligned sleep data and grind data is displayed with some graphical elements that present the time-aligned sleep data and grind data in a format that is easy to digest and that may be helpful in drawing some insights from the data that can be useful to the person.

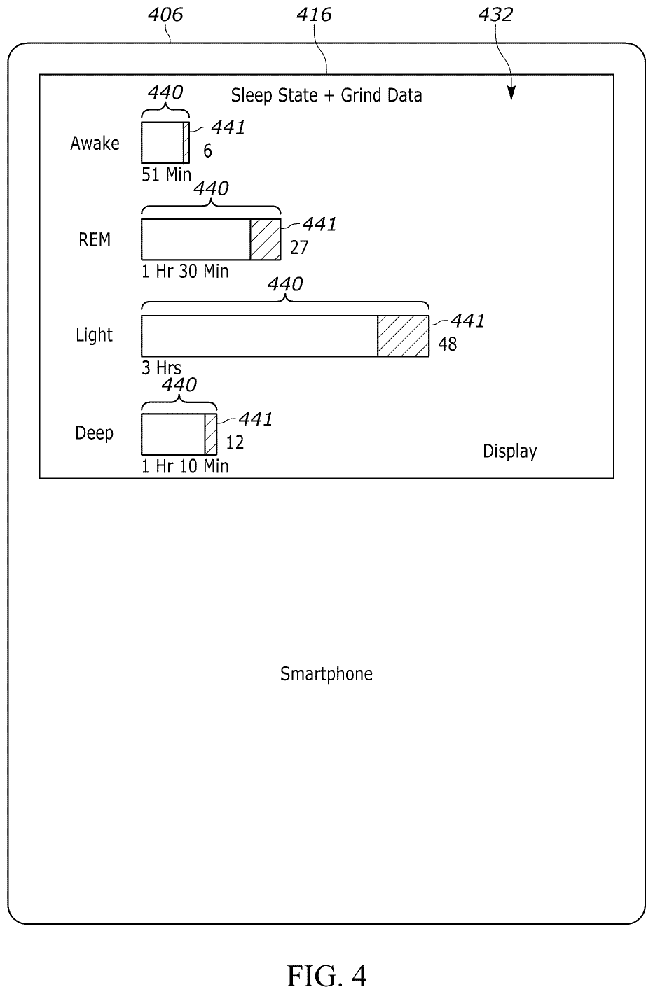

FIG. 4 is an example of a graphical representation of the time-aligned sleep data and grind data 432 in the form of a bar graph with horizontal bars for each of four sleep stages, light, deep, REM, and awake. The graphical representation is displayed on the display 416 of the smartphone 406. In the example, bars 440 of the bar graph have components that correspond to the total amount of time spent in each sleep stage for the respective night and components (e.g., the cross-hatched portion 441) that correspond to the total number of grinds counted while the person was in the respective sleep stage. For example, the awake bar represents 51 minutes of awake time and 6 grinds, the REM bar represents 1 hour 30 minutes REM sleep time and 27 grinds, the light bar represents 3 hours of light sleep time and 48 grinds, the deep bar represents 1 hour and 10 minutes of deep sleep time and 12 grinds. Thus, the graphical representation of the sleep data and the grind data is time-aligned at least because the number of grinds associated with a bar were counted while the person was in the particular sleep state. In the example, the width of the sleep stage component of a bar corresponds to the total length of time spent in that sleep stage over a night of sleep and the width of the grind count component of the bar corresponds to the total number of grinds counted while the person was in that sleep stage. The graphical representation may also include text and/or numbers identifying specific information, such as the length of time the person spent in each sleep stage, the percentage of the total time spent in each sleep stage, and/or the specific number of grinds that were counted in each sleep stage. As shown in FIG. 4, the number of grinds associated with a bar corresponds directly to a particular sleep stage such that the relationship between the number of grinds and a sleep stage is easily understood. Additionally, although the grind data is described in terms of a number of grinds (e.g., a grind count), the grind data may be presented on the display in other ways, e.g., by grind scores, or grind events, as described above.

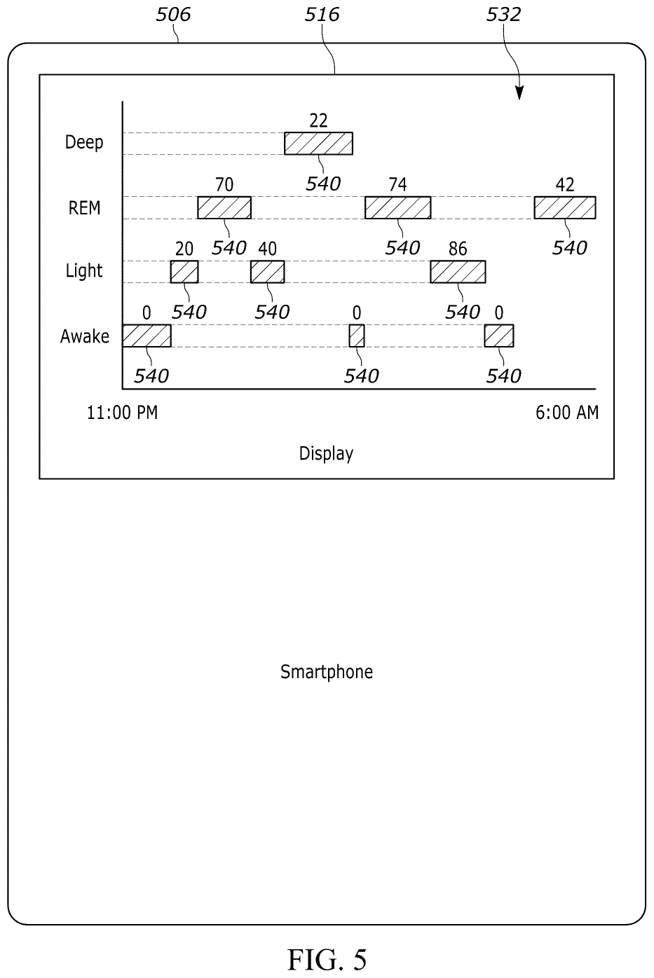

FIG. 5 is another example of a graphical representation of the time-aligned sleep data and grind data 532 in the form of a bar graph with horizontal bars that indicate an amount of time spent in a particular sleep stage as well as the particular time interval of that sleep stage. The graphical representation is displayed on the display 516 of the smartphone 506. As indicated on the graphical representation of FIG. 5, time is on the x-axis and sleep stage is on the y-axis. The graphical representation also includes a numerical indication of the grinding that occurred during the respective interval of sleep. For example, the numerical indication of the grinding may represent a number of grinds, a grind score, or a number of grind events, which corresponds to a magnitude or intensity of grinding. From the graphical representation of the time-aligned sleep data and grind data, it is easy for a person to understand how the magnitude or intensity of grinding (e.g., in terms of number of grinds, a grind score, or number of grind events) corresponds to sleep stage and to visualize how the prevalence of grinding unfolds throughout the night relative to the sleep stages.

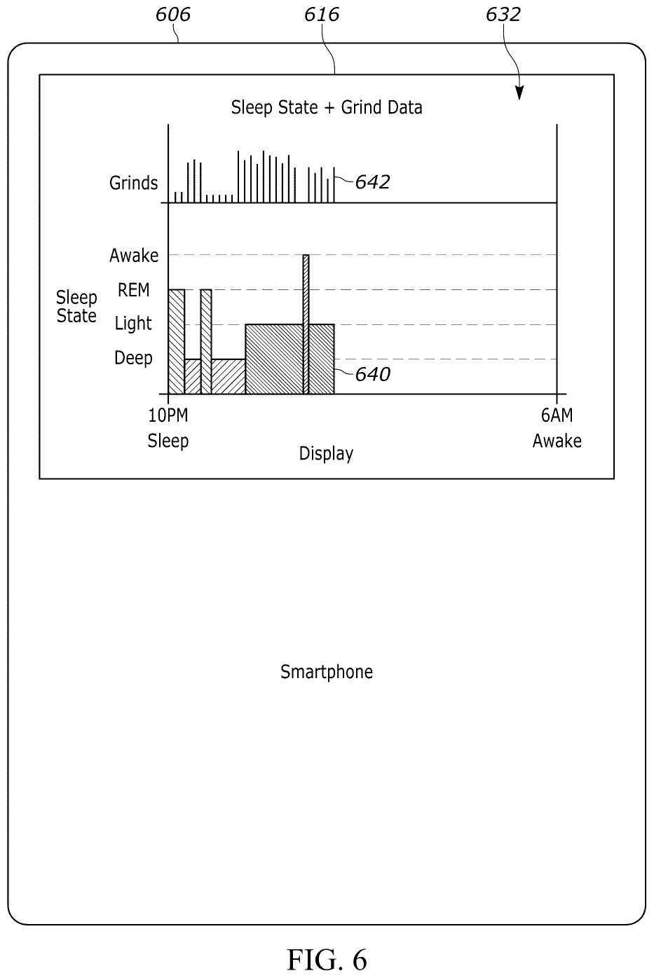

FIG. 6 is another example of a graphical representation of the time-aligned sleep data and grind data 632 in the form of a bar graph with vertical bars 640 that indicate sleep stage and vertical bars that indicate a magnitude of grinding (e.g., in terms of number of grinds, a grind score, or number of grind events) in which both sets of vertical bars are time-aligned with each other. The graphical representation is displayed on the display 616 of the smartphone 606. In the example of FIG. 6, the height of the sleep stage bars indicate the sleep stage (e.g., light, deep, REM, awake), and the width of the sleep stage bars indicate a length of time spent in that sleep stage (e.g., including a start time and end time), and the location of the sleep stage bars indicate the time during which the person was in the sleep stage, and the height of grind count bars indicates the number of grinds and the position of the grind count bars indicate the time at which the grinds occurred. From the graphical representation of the time-aligned sleep data and grind data it is easy for a person to understand how the number of grinds corresponds to sleep stages and to visualize how the prevalence of grinding unfolds throughout the night relative to the sleep stages.

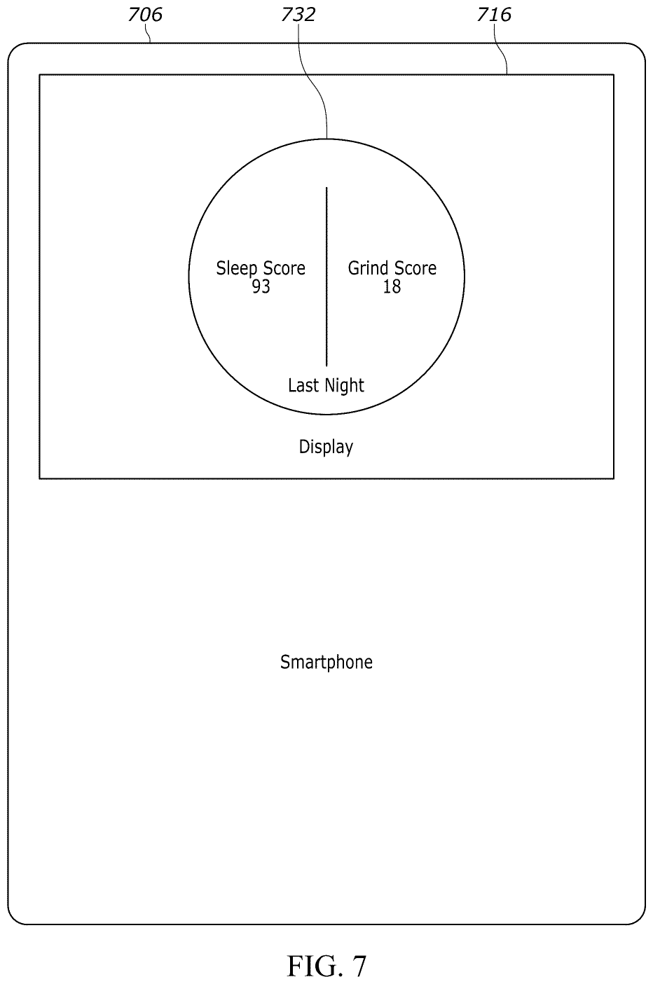

FIG. 7 is another graphical representation of the time-aligned sleep data and grind data 732 in the form of a sleep score and a grind score that are obtained from the sleep data and the grind data as described with reference to FIGS. 3A and 3B, respectively. The graphical representation is displayed on the display 716 of the smartphone 706. In the example, the sleep score may be generated from the collected heart rate data in various different ways. For example, the sleep score may be calculated as a function of the total time of sleep and/or a distribution of the time spent in particular stages. Likewise, the grind score may be generated from the collected motion data in various different ways. For example, the grind score may simply be a count of total grinds throughout an entire night of sleep or the grind score may be calculated as a function of the total number of grinds and/or a distribution of grinds throughout the night, and/or how the grinds correspond to the sleep stages. In the example of FIG. 7, the sleep data, which is displayed as a numeric sleep score, and the grind data, which is displayed as a numeric grind score, are time-aligned with each other in that the sleep score and the grind score were generated from heart rate data and from motion data that were collected over an overlapping time interval.

Although some examples of graphical representations of time-aligned sleep data and grind data are provided, other examples of graphical representations of time-aligned sleep data and grind data are possible. That is, it is expected that there are multiple different ways to graphically represent time-aligned sleep data and grind data that can provide a person with an easy way to understand the relationship between bruxism and sleep.

Because bruxism often involves a person involuntarily grinding their teeth while sleeping, a night guard or a dental splint (also referred to as an occlusal splint) may be worn to protect the teeth from the deleterious effects of grinding. Certain specially designed dental splints are also thought to reduce or eliminate the occurrence of bruxism. Although certain specially designed dental splints may reduce or eliminate the occurrence of bruxism, the grinding associated with bruxism typically occurs at night while a person is sleeping so it is difficult for the person to know if the occurrence of bruxism has been reduced or eliminated due to the use of a dental splint. Additionally, it is difficult for the person to know if any reduction in bruxism corresponds to any other benefits such as an improvement in the quantity and/or quality of their sleep.

As described above with reference to FIGS. 1-7, it can be beneficial to display a graphical representation of time-aligned sleep data and grind data on the display of a computer device. For example, displaying time-aligned sleep data and grind data provides a person with an easy way to understand the relationship between bruxism and sleep for their own personal health journey. However, such a display of time-aligned sleep data and grind day may not provide insights into the effectiveness of wearing a dental splint. However, it has been realized that a comparison between 1) time-aligned sleep data and grind data corresponding to a period when a dental splint was not being worn by a person, and 2) time-aligned sleep data and grind data corresponding to a period when a dental splint was being worn by the person can be displayed on a display of a computer device to provide a person with an easy way to understand how wearing the dental splint while sleeping or not wearing the dental splint while sleeping effects both their sleep and the occurrence of bruxism. Such information displayed to a user in an intuitive way can be an invaluable tool for managing their own personal health journey. In particular, it is believed that understanding the relationship between sleep and bruxism and the wearing or not wearing of a dental splint can be an invaluable tool in encouraging a person to consistently wear a dental splint. In an example, sleep data is generated from heart rate data collected from a wearable heart rate sensor and is associated with a splint presence indicator (SPI) that corresponds to when the heart rate data was collected from the wearable heart rate sensor, grind data is generated from motion data collected from a wearable motion sensor and is associated with a splint presence indicator that corresponds to when the motion data was collected from the wearable motion sensor, and the comparison of time-aligned sleep data and grind data is generated using the associated splint presence indicators corresponding to the sleep data and to the grind data.

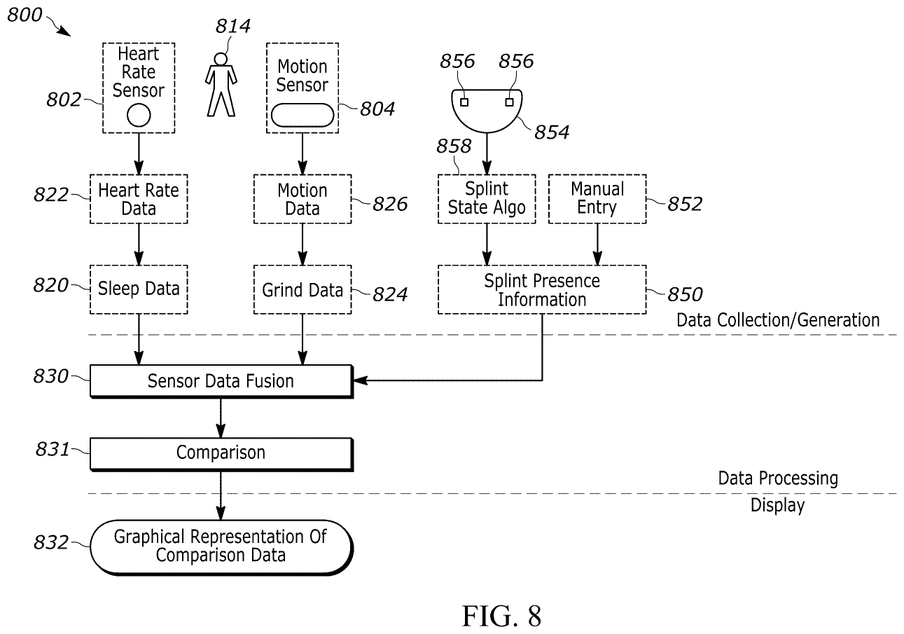

An example of a process for generating comparison data is described below with reference to FIGS. 8-10.

FIG. 8 illustrates an example of a process 800 for generating comparison data that can be displayed on the display of a computer device such as a smartphone. With regard to the sleep data 820, heart rate data 822 is generated from a heart rate sensor 802 of a wearable sleep tracking device that is worn by a person 814. The heart rate data that is collected by the heart rate sensor can be used to generate sleep data, for example, as described above. With regard to the grind data 824, motion data 826 is generated from a motion sensor 804 that is worn by the person 814. The motion data that is collected by the motion sensor can be used to generate grind data, for example, as described above.

In the example of FIG. 8, an additional parameter referred to herein as a splint presence information 850 is added to the sleep data and to the grind data in a sensor data fusion process 830. In an example, the splint presence information indicates whether or not the person was using a dental splint during a particular time interval. The splint presence information can be matched to sleep data 820 and grind data 824 to indicate whether or not the person was using a dental splint while the heart rate data was collected and while the motion data was collected. As mentioned above, a person may use a dental splint while sleeping to protect their teeth from damage due to grinding and/or to alleviate the grinding altogether. In order to evaluate how a dental splint affects sleep and grinding, it is important to know if the sleep data and the grind data correspond to the dental splint being used by (e.g., worn by) the person or the dental splint not being used by (e.g., worn by) the person. In one example, a user is prompted by a grind App to manually declare (e.g., manual entry 852) whether or not a dental splint was used during a particular night of sleep. For example, a user may be prompted by the grind App with a yes or no question each morning upon waking and engaging their smartphone, such as “Did you wear your dental splint last night?”. The answer to the question may be translated by the splint App into splint presence information for a particular time interval. In an example, splint presence information may include a start time, an end time, and a splint presence indicator (e.g., SPI=0: splint not in use; SPI=1: splint in use). An example of splint presence information is:

$$2023 - 10 - 31 T 20 : 00 : 00 : 2023 - 11 - 01 T 08 : 00 : 00 SPI = 0 / 1$$

The above example of splint presence information covers the time interval from 8 pm→8 am over which the person is assumed to have been sleeping and includes a value for SPI, in which SPI=0 for splint not in use during the time interval, and SPI=1 for splint in use during the time interval.

In another example, whether or not a dental splint was being worn is automatically determined. For example, a case 854 for the dental splint may be equipped with splint presence sensors 856 that can sense whether the dental splint is present in the case or not present in the case. From the output of the splint presence sensors, a splint state algorithm 858 executing on a processor embedded within the case can predict whether or not a dental splint was being used over a particular time interval. In an example, a splint state algorithm may interpret information generated from the splint presence sensors that are embedded within the case for a dental splint. For example, the splint state algorithm may set a splint presence indicator to SPI=0 when the splint presence information indicates that a dental splint was in the case for a time interval that includes typical sleeping hours, e.g., anywhere from 8 PM in the evening 8 AM the next morning and the splint state algorithm may set a splint presence indicator to SPI=1 when the splint presence information indicates that the dental splint was not in the case for a time interval that includes typical sleeping hours, e.g., anywhere from 8 PM in the evening 8 AM the next morning.

As illustrated in FIG. 8, sleep data 820, grind data 824, and splint presence information 850 (e.g., in the form of a time interval and a corresponding SPI) are used in a sensor data fusion process 830 to generate data that is used in a comparison process 831 to generate comparison data that can be graphically displayed to user. An example sensor data fusion process for generating time-aligned sleep data and grind data that includes splint presence information involves matching up in time a set of sleep data, a set of grind data, and a splint presence indicator.

As described above, a set of sleep data may include time-series sleep data that covers a particular time interval and a set of grind data may include time-series grind data that covers an overlapping time interval. Likewise, splint presence information may have a time interval identified by a start time and an end time and a corresponding SPI. In a sensor data fusion process, the sleep data, the grind data, and the splint presence indicator are aggregated into a time-aligned set of sleep data, grind data, and a splint presence indicator for each sleep event, where a sleep event is a time interval over which the sleep data indicates that the person was sleeping.

In one example, a set of time-aligned sleep data and grind data is generated for a single night of sleep as described with reference to FIGS. 3A-3D. The time-aligned sleep data and grind data is then matched with splint presence information that has an overlapping time interval. In an example, the matching of time-aligned sleep data and grind data with splint presence information can be expressed as:

$$2023 - 10 - 31 T 23 : 37 : 30 : 2023 - 11 - 01 T 05 : 31 : 00 : \text{} sleep / grind data ( time - aligned sleep data and grind data ) + 2023 - 10 - 31 T 20 : 00 : 00 : 2023 - 11 - 01 T 08 : 00 : 00 : SPI = 0 ( sleep presence information ) = 2023 - 10 - 31 T 23 : 37 : 30 : 2023 - 11 - 01 T 05 : 31 : 00 : sleep / grind data , \\ SPI = 0 ( time - aligned sleep data and grind data with SPI )$$

As expressed above, a set of time-aligned sleep data and grind data covers a time interval from 23:37:30 on Oct. 31, 2023 to 05:31:00 on Nov. 1, 2023 and includes a set of data as described with reference to FIG. 3D. The time interval of the set of time-aligned sleep data and grind data is then matched in time with a set of sleep presence information, in this case the set of sleep presence information that covers a time interval from 20:00:00 on Oct. 31, 2023 to 08:00:00 on Nov. 1, 2023. Because the two time intervals overlap, the SPI corresponding to the sleep presence information can be assigned to the set of time-aligned sleep data and grind data, which SPI indicates whether or not a dental splint was used by (e.g., worn by) the person during the time interval of the time-aligned sleep data and grind data.

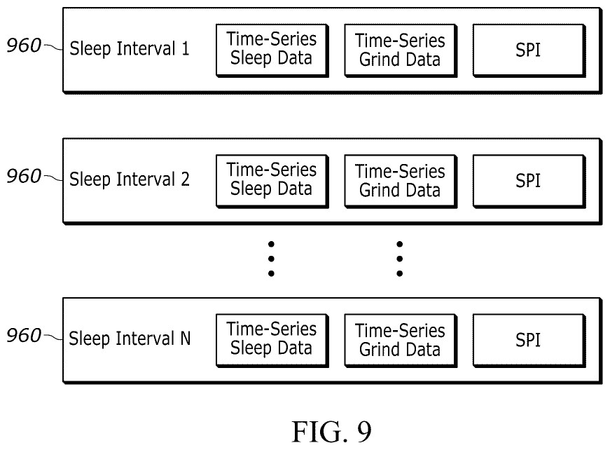

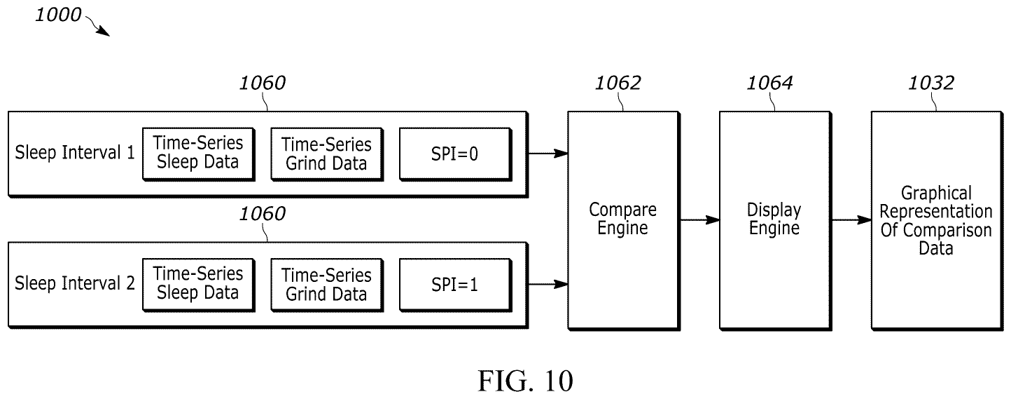

Sets of time-aligned sleep data, grind data, and a splint presence indicator are collected over multiple sleep events, e.g., over multiple different nights of sleep by the grind App and/or the grind App server. FIG. 9 depicts an example of multiple sets 860 of time-aligned sleep data, grind data, and a splint presence indicator that correspond to multiple different nights of sleep for a person. In an example, each set of data includes a set of data similar to that described with reference to FIG. 3D with the addition of an SPI that corresponds to the entire set of data. For example, each set of data represents a different night of sleep, including nights 1-N, where N is an integer greater than one. The data described with reference to FIG. 9 can be used in a comparison process to compare time-aligned sleep data and grind data collected when a person is wearing a dental splint (e.g., SPI=1) to time-aligned sleep data and grind data collected when the person is not wearing the dental splint (e.g., SPI=0). In an example operation, a data set that has a splint presence indicator that indicates a dental splint was used during the sleep interval (e.g., SPI=1) can be compared to a data set that has a splint presence indicator that indicates a dental splint was not used during the sleep interval (e.g., SPI=0) by looking at the SPIs of the sets of data to obtain a set of data with SPI=1 and a set of data with SPI=0. FIG. 10 illustrates an example of a process 1000 for generating a graphical representation of comparison data. As illustrated in FIG. 10, a data set 1060 that indicates a dental splint was used during the sleep interval (e.g., SPI=1) is compared to a data set 1060 that has a splint presence indicator that indicates a dental splint was not used during the sleep interval (e.g., SPI=0) by a compare engine 1062 and comparison data generated by the compare engine is used by a display engine 1064 to generate a graphical presentation of the comparison data. In an example, the compare engine and the display engine may include computing resources of the grind App server and/or the smartphone.

As described above, sleep data and grind data may be difficult to understand in a raw data format such as that shown in FIG. 9. A similar difficulty exists when trying to understand the effects of wearing a dental splint on sleep and grinding versus not wearing a dental splint. Thus, it has been realized that displaying a graphical representation of a comparison between 1) time-aligned sleep data and grind data corresponding to a period when a dental splint was not being worn by a person, and 2) time-aligned sleep data and grind data corresponding to a period when a dental splint was being worn by the person can be very helpful in understanding how the use of a dental splint affects sleep and/or grinding.

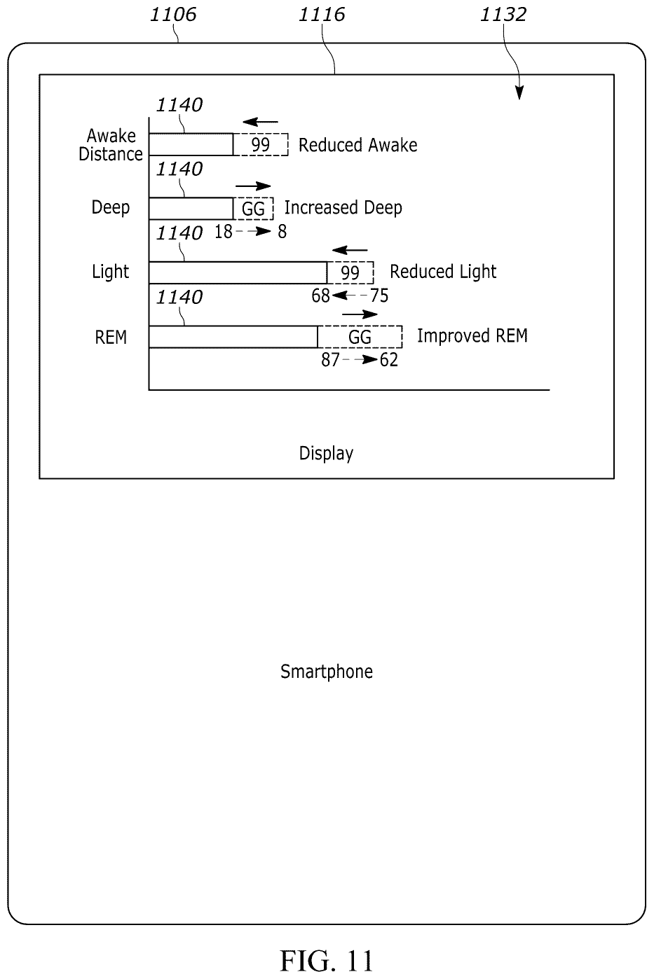

FIG. 11 is an example of a graphical representation 1132 of comparison data that includes a comparison between 1) time-aligned sleep data and grind data corresponding to a period when a dental splint was not being worn by a person (e.g., SPI=0), and 2) time-aligned sleep data and grind data corresponding to a period when a dental splint was being worn by the person (e.g., SPI=1). In the example, sleep data is generated from heart rate data collected from a wearable heart rate sensor and is associated with a splint presence indicator (SPI) that corresponds to when the heart rate data was collected from the wearable heart rate sensor, grind data is generated from motion data collected from a wearable motion sensor and is associated with a splint presence indicator that corresponds to when the motion data was collected from the wearable motion sensor, and the comparison of time-aligned sleep data and grind data is generated using the associated splint presence indicators corresponding to the sleep data and to the grind data, for example, as described with reference to FIG. 10. The graphical representation is displayed on the display 1116 of the smartphone 1106. In the example, the horizontal bars have components that correspond to a change in the total amount of time spent in each sleep stage between a night when a dental splint was not being worn by a person and a night when a dental splint was being worn by the person. In the example, the end portion of each bar corresponds to a change (either an increase or a decrease) in the total amount of time spent in the corresponding sleep stage between at least two different nights of sleep, with an arrow indicating whether the change is an increase or a decrease in the amount of time spent in the corresponding sleep stage. The graphical representation also includes lettering within the bar that further indicates whether the change was an increase (e.g., uppercase “GG” for “grind guard”) or whether the change was a decrease (e.g., lowercase “gg” for “grind guard”). The graphical representation also includes graphics in the form of numbers and arrows that indicate a change in the number of grinds for each sleep stage between a night when a dental splint was not being worn by a person and a night when a dental splint was being worn by the person. For example, in the deep sleep stage, the graphics includes “18→8”, which indicates that the number of grinds counted on a night when a dental splint was not being worn by the person was 18 and the number of grinds counted on a night when the dental splint was being worn by the person was 8. Likewise, in the light sleep stage, the graphics includes “68←75”, which indicates that the number of grinds counted on a night when the dental splint was not being worn by the person was 75 and the number of grinds counted on a night when the dental splint was being worn by the person was 68, and in the REM sleep stage, the graphics includes “87→62”, which indicates that the number of grinds counted on a night when the dental splint was not being worn by the person was 87 and the number of grinds counted on a night when the dental splint was being worn by the person was 62. Although the grind data is described in terms of a number of grinds (e.g., a grind count), the grind data may be presented on the display in other ways, e.g., by grind scores, or grind events, as described above. Displaying the comparison data as described with reference to FIG. 11 can provide a person with an easy way to understand how wearing the dental splint while sleeping or not wearing the dental splint while sleeping effects both their sleep and the occurrence of bruxism. Such information displayed to a user in an intuitive way can be an invaluable tool for managing their own personal health journey. For example, understanding the relationship between sleep and bruxism and the wearing or not wearing of a dental splint can be an invaluable tool in encouraging a person to consistently wear a dental splint.

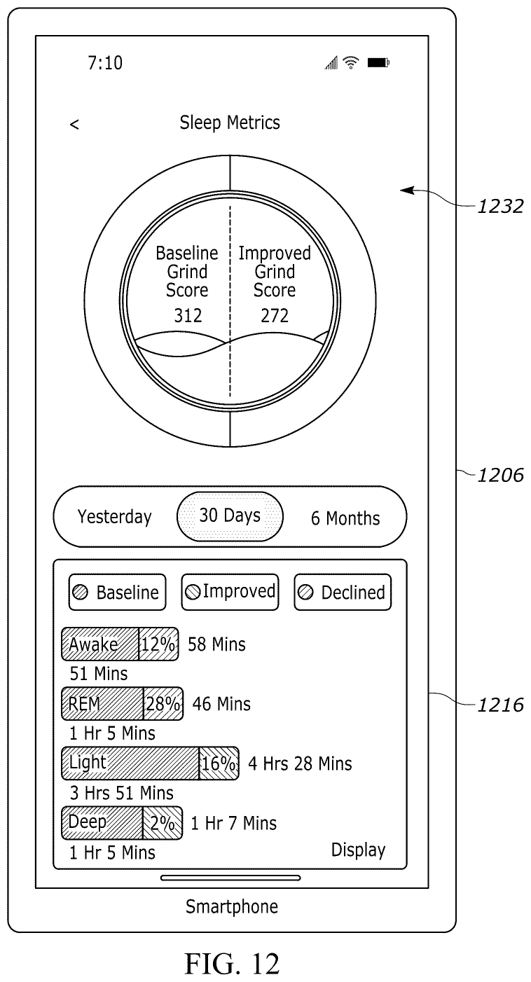

FIG. 12 is an example of a graphical representation 1232 of comparison data that includes a comparison between 1) time-aligned sleep data and grind data corresponding to a period when a dental splint was not being worn by a person (e.g., SPI=0), and 2) time-aligned sleep data and grind data corresponding to a period when a dental splint was being worn by the person (e.g., SPI=1). In an example, sleep data is generated from heart rate data collected from a wearable heart rate sensor and is associated with a splint presence indicator (SPI) that corresponds to when the heart rate data was collected from the wearable heart rate sensor, grind data is generated from motion data collected from a wearable motion sensor and is associated with a splint presence indicator that corresponds to when the motion data was collected from the wearable motion sensor, and the comparison of time-aligned sleep data and grind data is generated using the associated splint presence indicators corresponding to the sleep data and to the grind data, for example, as described with reference to FIG. 10. The graphical representation is displayed on the display 1216 of the smartphone 1206. In the example, the graphical representation includes a baseline grind score and an improved grind score that are displayed next to each other as numerical values. In an example, the baseline grind score is generated for a night of sleep in which a dental splint was not used by the person (e.g., as indicated by SPI=0) and the improved grind score is generated for a night of sleep in which the dental splint was used (e.g., as indicated by SPI=1). The grind scores may be generated from the collected motion data in various different ways. For example, the grind scores may simply be a count of total grinds throughout an entire night of sleep or the grind score may be calculated as a function of the total number of grinds and/or a distribution of grinds throughout the night, and/or how the grinds correspond to the sleep stages. The graphical representation also includes a bar graph of sleep stages in which the bars have components that correspond to a change in the total amount of time spent in each sleep stage between a night when a dental splint was not being worn by a person and a night when a dental splint was being worn by the person. In the example, the end portion of each bar corresponds to a change (either an increase or a decrease) in the total amount of time spent in the corresponding sleep stage between at least two different nights of sleep, with different hatching indicating whether the change is an increase or a decrease in the amount of time spent in the corresponding sleep stage. Note, in another example, color is used instead of different hatching to convey with a change was an increase or a decrease. For example, red color may be used instead of a first hatching to indicate a reduction in time and green color may be used instead of a second hatching to indicate an increase in time. In the example, the area of no hatching in each bar is considered as a baseline magnitude (e.g., when SPI=0). As shown in FIG. 12, the graphical representation may also include a key, which indicates that meaning of the hatching, e.g., baseline, improved, or declined. It should be noted that the key could also use colors that match to colors in the bar graph. Displaying the comparison data as described with reference to FIG. 12 can provide a person with an easy way to understand how wearing the dental splint while sleeping or not wearing the dental splint while sleeping effects both their sleep and the occurrence of bruxism. Such information displayed to a user in an intuitive way can be an invaluable tool for managing their own personal health journey. For example, understanding the relationship between sleep and bruxism and the wearing or not wearing of a dental splint can be an invaluable tool in encouraging a person to consistently wear a dental splint.

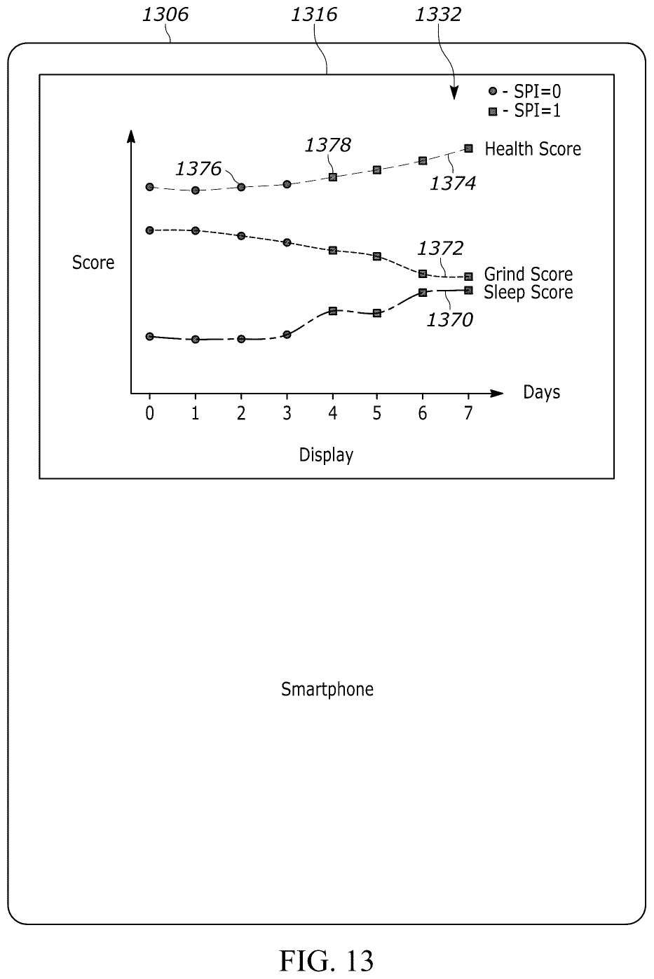

FIG. 13 is another example of a graphical representation 1332 of comparison data that includes a comparison between 1) time-aligned sleep data and grind data corresponding to a period when a dental splint was not being worn by a person (e.g., SPI=0), and 2) time-aligned sleep data and grind data corresponding to a period when a dental splint was being worn by the person (e.g., SPI=1). In an example, sleep data is generated from heart rate data collected from a wearable heart rate sensor and is associated with a splint presence indicator (SPI) that corresponds to when the heart rate data was collected from the wearable heart rate sensor, grind data is generated from motion data collected from a wearable motion sensor and is associated with a splint presence indicator that corresponds to when the motion data was collected from the wearable motion sensor, and the comparison of time-aligned sleep data and grind data is generated using the associated splint presence indicators corresponding to the sleep data and to the grind data, for example, as described with reference to FIG. 10. The graphical representation is displayed on the display 1316 of the smartphone 1306. In the example, the graphical representation includes plots of three scores on the y-axis vs. days on the x-axis, with the three plots being a sleep score plot 1370, a grind score plot 1372, and a health score plot 1374 and in which each data point is marked by an element with a shape that identifies whether the data point corresponds to the dental splint having been used for that night (e.g., SPI=1) or to the dental splint having not been used for that night (e.g., SPI=0). For example, data points that correspond to SPI=0 are represented by circular elements 1376 and data points that corresponds to SPI=1 are represented by square elements 1378. In the example, the sleep score may be generated from the collected heart rate data in various different ways. For example, the sleep score may be calculated as a function of the total time of sleep and/or a distribution of the time spent in particular stages. Likewise, the grind score may be generated from the collected motion data in various different ways. For example, the grind score may simply be a count of total grinds throughout an entire night of sleep or the grind score may be calculated as a function of the total number of grinds and/or a distribution of grinds throughout the night, and/or how the grinds correspond to the sleep stages. In the example, it is desirable to increase the sleep score and decrease the grind score, especially over a period of days in which a dental splint is being used. In the example, the “health score” is generated as a function of the sleep score and the grind score in which the health score increases (improves) as the sleep score increases and as the grind score decreases. The graphical representation can provide an easy way to understand how wearing the dental splint while sleeping or not wearing the dental splint while sleeping effects both their sleep and the occurrence of bruxism over multiple days, weeks, or months.

Although some examples of graphical representations of comparison data have been described, other examples are possible.

In some examples, sleep stages are defined in terms of “REM” and “NREM,” where REM sleep stands for “rapid eye movement” and can also be called “stage R” and NREM sleep (or non-rapid eye movement) sleep includes light and deep sleep stages, and may also be referred to NREM stages 1-4, with light sleep being NREM stages 1-2 and deep sleep encompassing NREM stages 3-4. In an example, Non-REM 1 is between waking and sleep, Non-REM 2 is light sleep, which is shallow and choppy, Non-REM 3 is deep sleep, which is harder to wake from and can help with muscle recovery, and REM is when most dreaming occurs. In some examples, the sleep stages are labeled as light, deep, REM, and awake. However, other labels and/or other sleep stages may be used. Additionally, other terms may be used to identify a similar sleep stage. For example “light” sleep may be referred to as “core” sleep and deep sleep may be referred to as “Slow Wave Sleep” or as “SWS”.

In an example, a graphical representation of time-aligned sleep data and grind data may include multiple graphical elements that combine to form a “graphic” or a “graphical representation.” In an example, a graphical element may include a symbol, symbols, a word, words, and/or colors.

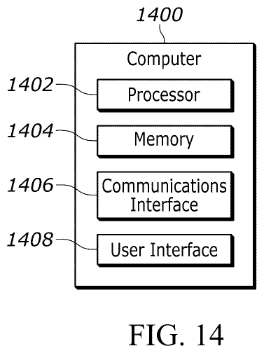

In an embodiment, the above-described functionality is performed at least in part by a computer or computers (e.g., a host computer and/or a processor of a NIC), which executes computer readable instructions. FIG. 14 depicts a computer device 1400 such as a smartphone that includes a processor 1402, memory 1404, a communications interface 1406, and a user interface 1408. The processor may include a multifunction processor and/or an application-specific processor. Examples of processors include the PowerPC™ family of processors by IBM and the x86 family of processors by Intel such as the Xeon™ family of processors and the Intel X5650 processor. The memory within the computer may include, for example, storage medium such as read only memory (ROM), flash memory, Random Access Memory (RAM), and a large capacity permanent storage device such as a hard disk drive. The communications interface enables communications with other computers via, for example, the Internet Protocol (IP). The user interface may include a display device, buttons, speaker, and/or microphone as is known in the field. The computer executes computer readable instructions stored in the storage medium to implement various tasks as described above.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

It should also be noted that at least some of the operations for the methods described herein may be implemented using software instructions stored on a computer useable storage medium for execution by a computer. As an example, an embodiment of a computer program product includes a computer useable storage medium to store a computer readable program.

The computer-useable or computer-readable storage medium can be an electronic, magnetic, optical, electromagnetic, infrared, or semiconductor system (or apparatus or device). Examples of non-transitory computer-useable and computer-readable medium include a semiconductor or solid state memory, magnetic tape, a removable computer diskette, a random access memory (RAM), a read-only memory (ROM), a rigid magnetic disk, and an optical disk. Current examples of optical disks include a compact disk with read only memory (CD-ROM), a compact disk with read/write (CD-R/W), and a digital video disk (DVD).

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A method comprising:
displaying, on a display of a computing device, time-aligned sleep data and grind data;
wherein;
the sleep data was generated from heart rate data collected from a wearable heart rate sensor; and
the grind data was generated from motion data collected from a wearable motion sensor.

2. The method of claim 1, wherein the sleep data is graphically represented in multiple different sleep stages on the display and the grind data is displayed on a sleep stage-specific basis.

3. The method of claim 1, wherein the sleep data is graphically represented in multiple different sleep stages on the display and the grind data is displayed on a per-sleep stage basis.

4. The method of claim 1, wherein the sleep data is graphically represented as sleep stages that include light, deep, REM, and awake and the grind data is displayed on a sleep stage-specific basis.

5. The method of claim 1, wherein the sleep data is graphically represented as sleep stages that include light, deep, REM, and awake and the grind data is displayed as a numerical representation of grinds per sleep stage.

6. The method of claim 1, wherein the time-aligned sleep data and grind data includes graphical indicators that link the sleep data to the grind data in time.

7. The method of claim 1, wherein:
the sleep data includes time-series sleep stage data;
the grind data includes time-series grind data;
the time-aligned sleep data and grind data is generated by matching times of the time-series sleep stage data with times of the time-series grind data.

8. The method of claim 7, wherein:
the time-series sleep stage data is displayed as sleep stages of light, deep, REM, and awake; and
the time-series grind data is displayed with a sleep stage that has a matching time.

9. The method of claim 7, wherein grind data from the time-series grind data is assigned to a sleep stage that has a matching time.

10. The method of claim 1, wherein the grind data corresponds to a number of grinds that are detected from the motion data.

11. The method of claim 1, wherein the grind data is displayed as a numerical representation of grinds per sleep stage.

12. The method of claim 1, wherein the grind data is displayed as a grind score per sleep stage.

13. The method of claim 1, wherein the grind data is displayed as a total number of grinds for a night of sleep.

14. The method of claim 1, wherein the grind data is displayed as a grind score for a night of sleep.

15. The method of claim 1, wherein the time-aligned sleep data and grind data were generated from heart rate data and motion data that were collected in time intervals that overlap with each other.

16. The method of claim 1, wherein the wearable heart rate sensor is a finger worn device and the wearable motion sensor is a chin worn device.

17. The method of claim 1, wherein the wearable heart rate sensor is a wrist worn device and the wearable motion sensor is a chin worn device.

18. A non-transitory computer readable medium comprising instructions to be executed in a computer system, wherein the instructions when executed in the computer system perform a method comprising:
displaying, on a display of a computing device, time-aligned sleep data and grind data;
wherein;
the sleep data was generated from heart rate data collected from a wearable heart rate sensor; and
the grind data was generated from motion data collected from a wearable motion sensor.

19. A system comprising:
a display;
a processor; and
memory with instructions stored thereon, wherein the instructions when executed by the processor cause time-aligned sleep data and grind data to be displayed on the display, wherein;
the sleep data was generated from heart rate data collected from a wearable heart rate sensor; and
the grind data was generated from motion data collected from a wearable motion sensor.
